(19) United States
(12) Patent Application Publication
(10) Pub. No.: US 2004/0039337 A1
(43) Pub. Date: Feb. 26, 2004

(54) Portable safety auto-injector

(76) Inventor: Michael Alexander Letzing, San Francisco, CA (US)

Correspondence Address: Michael Letzing, 2251 Broadway, San Francisco, CA, 94115, US

(21) Appl. No.: 10/375,305

(22) Filed: Feb. 27, 2003

Related U.S. Application Data
(60) Provisional application No. 60/404,551, filed on Aug. 21, 2002.

(51) Int. Cl. A61M 5/20

(52) U.S. Cl. 604/157; 604/117

(57) ABSTRACT

An auto-injector body has a movable spring loaded hypodermic needle and attached plunger activated drug container. The auto-injector body is housed within a palm-sized handle and biased so that a sealed end of the auto-injector body protrudes from an open end of the handle. A cover encloses the protruding end and mates with the handle and has external attaching means such as a key chain, belt clip, or strap slots. Pressing the sealed end of the auto-injector body against the skin forces the auto-injector body into the handle to fire a trigger which releases a spring to force the needle through the sealed end into the user and force the plunger to inject the drug through the needle.

### CLAIM OF PROVISIONAL APPLICATION RIGHTS

This application claims the benefit of a United States Provisional Patent Application “Highly portable and durable auto-injector” (Application No. 60/404,551; Filing Date: Aug. 21, 2002).

### BACKGROUND OF THE INVENTION

1. Field of the Invention

This application relates to devices for injecting liquid medicaments and, more particularly, to a portable and durable auto injector which may be carried in a pocket and has a hard enclosed safety case and safety features that insure an accurate injection and prevent premature firing.

2. Description of the Prior Art

Allergies, such as those to food and bee-stings, can be a very dangerous affliction. In people who suffer from these allergies, a life threatening condition called anaphylaxis can develop in seconds after exposure to the allergen. If the condition is not treated immediately, their airway can soon close and death can result from suffocation.

One widely accepted treatment for anaphylaxis is a hypodermic injection of epinephrine. Usually, the patient carries and administers this medication through the use of an automatic injector. Due to the fast action of many allergens and the symptoms of anaphylaxis, the portability of the treatment mechanism is of utmost importance.

Automatic injectors are well known. Basically, an automatic injector is a device for enabling an individual to self-administer a dosage of a liquid medicament. An advantage of automatic injectors is that they contain a measured dosage of a liquid medicament in a sealed sterile condition capable of storage in such condition for an extensive period of non-use, during which period immediate injection of the stored dosage may be accomplished at any time under the most severe emergency conditions. Another advantage of automatic injectors is that the administration of the self-contained dosage of liquid medicament is accomplished without the necessity of the user initially seeing the hypodermic needle through which the liquid medicament is injected or of manually penetrating such a visible needle into the user's own tissue. Instead, an automatic injector includes a releasable stressed spring assembly. This assembly includes a stressed spring, a releasable mechanism for releasably retaining the spring in a stressed storage position and a releasing mechanism for releasing the releasable mechanism in response to a predetermined actuating procedure.

Automatic injectors have heretofore been particularly suited for use under emergency conditions. For example, many tens of millions of such automatic injectors have been manufactured and sold containing nerve gas antidotes for use under emergency chemical warfare conditions. Typical units which have been utilized for this purpose are disclosed in U.S. Pat. Nos. 2,832,339, 3,882,863, and 4,031,893. In addition, units of this type have been proposed for use in administering antiarrhythmic medicaments under emergency conditions relating to heart attack medical situations. Such use has been in conjunction with portable monitors as is evident from the disclosure contained in U.S. Pat. Nos. 3,910,260 and 4,004,577. It has also been proposed to provide other medicaments useful in treating heart attack symptoms such as clot selective thrombolytic agents (e.g. tPA) and related medicaments. See, for example, U.S. Pat. Nos. 4,689,042, 4,755,169, and 4,795,433. Finally, automatic injectors have been marketed in recent years containing a dosage of epinephrine as an antidote for counteracting severe allergic reactions, as for example, to bee stings and the like.

Currently available auto-injector devices are generally the size of a large fountain pen with the button at one end. They are operated by wrapping one's fingers around the shaft of the pen, holding it against the injection site, and then firing the injection using the thumb similar to using a click-style ball point pen

Patients frequently neglect to carry their medication with them. The reason for this is that the design of the auto-injector device is not as portable as it needs to be. Portability is one of the most important characteristics of an auto-injector. The times at which an auto-injector is most frequently needed usually coincide with the times when its portability are most important. Taking the bee-sting and food-allergy examples again, a bee-sting is most likely to happen when one is outside and participating in active pastimes. Carrying around an auto-injector in these circumstances requires great portability. Food-allergies normally take place in restaurants, where a person might encounter the food they are allergic to accidentally. Again in this circumstance, having an auto-injector requires great portability to get the patient to comply with carrying the device at all times.

The currently available auto-injectors are suitable to be kept in one's vehicle and one's home, but not in one's pocket. The length of the device is prohibitive in that it does not allow one to move or sit freely when placed in one's pant pocket.

Current pen-style auto-injection devices are difficult to use, since they are used by holding the device with one hand, pressing one end of it against the injection site (usually the leg), and then firing the device using the thumb (the trigger is located on the other end opposite the injection site). To visualize, picture holding an ordinary pen in your palm while pressing the writing tip against your leg and holding your thumb at the opposite end of the pen.

Current pen-style models demonstrate the desire for the device to be portable. However, due to the way in which the device is used, there is a limit as to how short or how small the auto-injector can be. Because it has to protrude from both sides of a closed fist, the device can be no shorter than the width of an above-average sized human fist (Otherwise, the needle would never reach the injection site and would not penetrate the skin). Therefore in order to have a device that is functional and solves the problem of size as described above, the design must be modified.

In addition to the length problem, making the device smaller takes any all the leverage that the pen-style auto-injectors afford. This leverage is necessary to hold the device against the site of injection while firing the device, which acts by a spring mechanism.

In prior auto-injector devices, misfires are possible because the device does not have to be positioned against the skin in order to fire. This presents a significant disadvantage in that it makes the chance of error by the user too when self-administering medication possibly leading to death of the user by not getting the medication.

What is needed is a pressure-sensitive auto-injector which must be positioned against the skin of the user and which would allow for a smaller device that still provides the leverage necessary to operate the device effectively. Provision of a sufficiently small and durable auto-injector is necessary to insure that people will always carry it with them.

### SUMMARY OF THE INVENTION

An object of the present invention is to provide a new smaller auto-injector, about the size of a tube of lipstick so that it can be conveniently carried in a pocket or a small purse.

Another object of the present invention is to provide an auto-injector which is more portable and more durable than existing models and has a hard waterproof case with attachments that can be attached to one's key-chain, clipped to one's belt, or attached with a Velcro band to an extremity or an object such as a bicycle. Since the key-chain, is the one thing that most people have with them at all times, it is an ideal vehicle for reliable transport of an auto-injector.

A further object of the present invention is that it has a built-in safety feature which requires the injector to be positioned against the injection site in order to fire which will allow less chance for error when the medication is being self-administered.

Still another object of the present invention is that it provides an auto-injector which requires pressure against the injection site to activate the device which insures proper placement against the skin to perform the injection and maintain the auto-injector in place during the injection.

One more object of the present invention is to provide an auto-injector wherein the needle is not exposed to view and a rubber membrane is pressed against the skin to push the injector portion into the case and enable a trigger to engage the end of the case and fire the spring-loaded auto-injector which inserts the needle into the skin through the membrane and injects the shot of medicine into the user, thereby maintaining a sterile environment for the needle and avoiding the trauma and trepidation associated with seeing the needle and having to insert the needle into oneself, thereby making it much easier and insuring greater accuracy in self-injecting.

Another object of the present invention is to provide an auto-injector which is simple in construction, effective in operation and economical to manufacture.

In brief, the auto-injector of the present invention represents a paradigm shift in the way an auto-injector device is activated and then fired. The present auto-injector basically consists of three main parts. The three parts of the device are the cover, the handle, and the auto-injector body. When fully assembled and in the safety position, the device is about the same size and shape as a tube of lipstick.

The cover is the simplest part of the device and basically acts as a durable shield preventing the device from being triggered accidentally. Using the lipstick analogy, the cover is analogous so the cover of a tube of lipstick. The main function of the cap is to prevent the accidental triggering of the auto-injector. Additionally, the cap prevents dirt and contaminants from coming in contact with the injection site, and also serves as the anchor point for the key-chain ring and the belt clip or slots or straps.

The cover is attached to the main device (handle & auto-injector body) either by a screw on or gas-cap-type screw on which will not tighten beyond a certain amount (so that it never gets too tight to open easily in an emergency) or friction pop-cap mechanism or any of a variety of child-proof-types of cover connections. A friction pop-off cap may be preferable to a screw design or child-proof-type connector so that the cap is easily removed and does not become tightly lodged onto the body, thus preventing the user from getting to the medication in an emergency. The cap can be attached in a way that prevents children from opening the cover. The most obvious method for doing this is the child-proof screw cap utilized in medication containers.

Any of the many child-proof-type connections could be used between the cover and the body. The cover could be removed by pushing it in towards the handle and then twisting. After twisting the cover, it would be easily removed by pulling it away from the main body. The main disadvantage to this design is that it might be difficult to get the cap off quickly in an emergency (especially due to the small size of the device). The cover could operate like the top on a container of medicine. It would have to be pushed in towards the handle and then screwed off. Again, this could be difficult to remove quickly in an emergency.

When the cap has been removed, the device is considered activated, and is ready for use as an auto-injector.

The handle is the part of the device that the user holds. Its main function is to provide the leverage necessary for a user to press the device in towards the skin, thus resulting in self-injection. It is basically a hollow tube, closed on one end with a recessed ring on the same closed end for gripping by the fingers. The other end is open. The finger grips are provided so that the user of the device can get a firm hold on the auto-injector. Within the handle is a hollow cavity. The cavity is designed of such diameter that the auto-injector body fits snugly inside of it, yet is free to slide into and out of the cavity. There is a lip at the open end of the cavity, which prevents the auto-injector from sliding completely out of the cavity. On the inside of the closed end of the cavity there is a spring. There is also a platform against which the trigger of the auto-injector body can press. The spring keeps a constant pressure against the auto-injector, preventing its trigger from coming into contact with the platform and thus being fired. However, when the user of the device presses the auto-injector body against the injection site, the force of the spring is overcome and the trigger comes into contact with the platform. This contact releases an internal spring within the auto-injector that causes the device to fire and the medication to be released.

The size of the auto-injector body is slightly smaller than that of the cover and handle and fits snugly inside when the device is in the safety position. The diameter is such that it can slide in and out of the cavity of the handle and that it is completely covered by the cap when the device is in the safety position.

The auto-injector body is bound on one side by a plastic edge and rubber sheath, which prevents the user from coming into accidental contact with the hypodermic needle and maintains the sterility of the needle. The hypodermic needle could alternatively be enveloped in a rubber sheath itself. Adjacent to the rubber membrane/sheath on the inside of the cavity is the hypodermic needle. A medicine cartridge is within the interior chamber and attached to the hypodermic needle. The medicine cartridge and needle are separated from each other by either a burstable seal or a one-way valve. The separation prevents the flow of liquid medicine out of the cartridge and into the needle cavity before the device is fired. (Some medications deteriorate when stored in contact with metal.)

When pressure is applied to the liquid within the medicine cartridge, however, the seal bursts (or valve opens) and the liquid flows into and then out of the hypodermic needle. The hypodermic needle and medicine cartridge can slide as a unit within the interior chamber of the auto-injector body. They are not free to slide through the rubber sheath and out of the cavity. Backward pressure from the plunger (discussed below) prevents this from happening until the device is fired.

The medicine cartridge is bound on the side opposite the hypodermic needle by a plunger. The plunger acts dually to both prevent the liquid medication from leaving the medicine chamber and to force the medicine out through the hypodermic needle when the device is fired. It also acts to hold the medicine cartridge and needle inside the sheath until the device is fired. Adjacent to the plunger is the releasable stressed spring assembly, which controls the firing of the device. A trigger is bound so the exterior of the device on the side opposite the rubber sheath. The trigger controls the releasable stressed spring assembly.

When the device is fired by the depression of the trigger, the stressed spring assembly is released, thus causing a constant force of the plunger. The force on the plunger causes the needle/medication cartridge device to be forced into the operative position (see x-section figure). In this position, the hypodermic needle has been forced through the rubber sheath and into the tissue of the user of the device by the pressure of the spring assembly on the plunger and thus also on the medicine cartridge/needle housing. The needle stops moving forward due to the stop wedges on either side of it, which eventually come so rest on the edge of the exterior housing. At this point force from the spring can cause only the plunger and not the cartridge/needle housing to move forward. The force of the needle on the plunger causes the plunger to move forward and thus the ejection of the medicine from the medical cartridge. The medicine flows through the seal or valve, and then into and out of the hypodermic needle.

The safety device or cover is first removed by unscrewing it from the base/handle. That act exposes the auto-injector body and thus the rubber membrane behind which the needle and medication lie. After having exposed this area, the handle of the device is grabbed in such any that the end of the device where the membrane resides protrudes from the hand. The suggested handling of the device is to grasp the handle such that the fingers wrap around the finger grips. The body of the device then will be protruding from the hand. This grasping technique will allow the user of the device to be able to apply enough pressure to cause the device to fire. Holding the device as said above, the rubber membrane is placed on the site of injection and then the device is pressed in against the injection site until it fires. At that point the auto-injector spring mechanism deploys the needle into the skin and ejects the prescribed dose of medication.

An advantage of the present invention is that it is much more portable than currently available models.

Another advantage of the present invention is that it is much more durable than currently available models and is waterproof.

Another advantage of the present invention is that it prevents dirt or other contaminants from coming into contact with the injection site and possibly leading to later infection.

A further advantage of the present invention is that it will allow less chance for error when the medication is being self-administered.

One more advantage of the present invention is that it is easy to self inject since the needle is not visible and the user never has to stick the needle in since it is all automatic upon pressing the device against the skin.

### BRIEF DESCRIPTION OF THE DRAWINGS

These and other details of my invention will be described in connection with the accompanying drawings, which are furnished only by way of illustration and not in limitation of the invention, and in which drawings:

FIG. 1 is a perspective view of the auto-injector of the present invention showing the auto-injector body held within and slightly protruding from the handle and the cover aligned for connection to the handle;

FIG. 2 is a cross-sectional view of the handle showing the auto-injector body in the handle in the storage position with the auto-injector body partially protruding from the handle opening where the cover will be connected to the handle to cover the protruding portion of the auto-injector body;

FIG. 3 is a cross-sectional view of the handle showing the auto-injector body in the handle in the injection position with the auto-injector body pushed into the handle by pressing the rubber membrane 41 against the body of the user and thereby causing the trigger to engage the end of the handle in a first position (shown dashed) thereby releasing the injector spring to push the needle through the rubber membrane and into the user and also inject the drug with the plunger forcing the drug through the end of the needle.

### BEST MODE FOR CARRYING OUT THE INVENTION

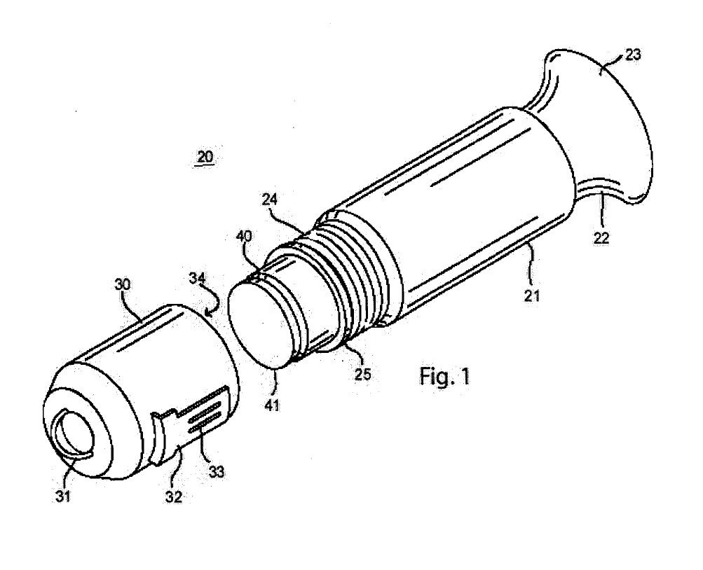

In FIGS. 1-3 an auto-injector device 20 fits into a pocket and has safety features to prevent premature firing and an injection feature which insures accurate injections.

A handle 21 is capable of fitting within a palm of a user and has a solid exterior shell with a single handle opening 18 at one end and a hollow interior cavity 19 communicating with the handle opening. The handle 21 has a recessed ring 22 with an end ridge 23 around its periphery for gripping.

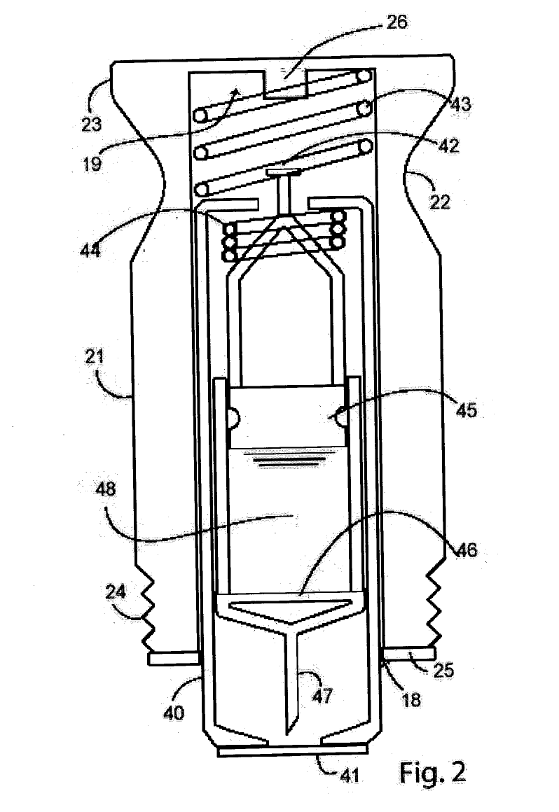

An auto-injector body 40 is housed slidably within the hollow interior cavity 19 of the handle 21 and has a sealed end portion, preferably with a rubber membrane 41, protruding out of the handle opening, as seen in FIGS. 1 and 2. The auto-injector body 40 houses a movable hypodermic needle 47 and attached drug container assembly, including a drug container chamber 48 with a plunger 45.

The handle 21 has a closed end 26 opposite to the end opening 18 and further comprises a normally extended spring 43, as seen in FIG. 2, between the closed handle end 26 and the inner end of the auto-injector body 40. The normally extended spring 43 biases the auto-injector body 40 so that the sealed end protrudes out of the handle and the normally extended spring means is capable of yielding upon a pressing in of the auto-injector body 40 so that the trigger 42 contacts the interior of the closed end 26 of the handle 21.

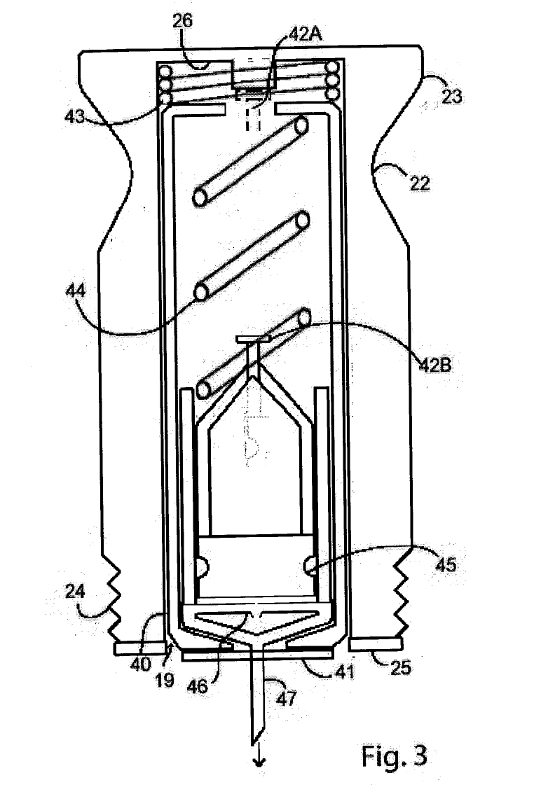

A normally compressed spring 44, as seen in FIG. 2, between the closed end of the auto-injector body and a top of the plunger 45 serves as a needle moving means and a drug injecting means. A trigger mechanism, such as a trigger 42 at the inner end of the plunger 45, activates the spring 44 with a restraining means normally used for such devices (not shown) Upon pressing the sealed end portion with rubber membrane 41 against a body of a user, the auto-injector body 40 is forced into the handle 21 to activate the trigger 42A (shown dashed) by contact of the trigger 42A with the interior closed end 26 of the handle, as shown in FIG. 3, and the trigger mechanism releases the restraining means and causes the normally compressed spring 44 to be released, as seen in FIG. 3, and force the needle 47 through the sealed end portion 41 and into the body of the user (not shown) and force the plunger 45 to eject the drug from the drug chamber 48 and out through the needle 47 (arrow in FIG. 3) and inject the drug into the body of the user.

A cap 30, as seen in FIG. 1, has a solid shell and a handle receiving opening 34 in one end capable of receiving and securing the handle 21 therein, by screw threads 24 and a rubber seal 25 or other means such as a friction fit or child-proof closure, with the cover around the handle end opening and a hollow interior cover cavity (not shown) capable of receiving and shielding the sealed end portion of the auto-injector body 40 therein.

The cover 30 further comprises a means for attaching the cover to an external object, including a key attaching means, such as an end loop 31 to receive a keychain, a clip 32 for attaching to clothing or a belt, and a pair of parallel slots 33, or other slot means, to receive a strap therethrough for attaching to other objects, such as a Velcro tie strap for attaching to a bicycle frame or other objects.

While the preferred embodiment has been described other variations include a cap attached to the handle with a gas cap type screw which cannot be tightened beyond a certain amount to insure that a user will be able to unscrew it in an emergency. A childproof cap might comprise a press and screw or squeeze and screw cover. Variations of the auto-injector activator include various locations for the trigger including a protective cover for the trigger if it is outside the handle to prevent unintentional activation.

Variations of the firing mechanism are possible within the scope of the palm-sized device claimed. A trigger, or button, could be placed on the side of the device such that when the button is pressed the needle is fired. In fact, the trigger could really be placed anywhere on the device, and then protected or child-proofed with some kind of covering, cap, or sheath. With the trigger on the side, the device could be held against the leg and then squeezed until the trigger is depressed and auto-injection occurs. The main disadvantage is that the device is prone to being misfired. When the user holds it, they could accidentally squeeze too hard and activate the device before they are ready.

There are other possible variations of the auto-injector, including a reusable auto-injector wherein the drug can be reloaded into the device, an auto-injector providing multiple injections (the user can administer more than one shot with the same device without reloading), an auto-injector wherein the medicine is in contact with the needle and another wherein the medicine is in a compartment that is not in contact with the needle. (Some medications break down in the presence of metals, which the needle is made of), and an auto-injector wherein two or more liquids are injected at once that can not be in contact with one another for prolonged periods.

It is understood that the preceding description is given merely by way of illustration and not in limitation of the invention and that various modifications may be made thereto without departing from the spirit of the invention as claimed.

What is claimed is:

1. An auto-injector device which fits into a pocket and has safety features to prevent premature firing and an injection feature which insures accurate injections, the device comprising:
a handle capable of fitting within a palm of a user and having a solid exterior shell with a single handle opening at one end and a hollow interior cavity communicating with the handle opening;
an auto-injector body capable of being housed slidably within the hollow interior cavity of the handle and having a sealed end portion protruding out of the handle opening, the auto-injector body housing a movable hypodermic needle and attached drug container assembly, a needle moving means and a drug injecting means and a trigger mechanism therein so that upon pressing the sealed end portion against a body of a user, the auto-injector body is capable of being forced into the handle to activate the trigger mechanism and the trigger mechanism is capable of causing the needle moving means and drug injecting means to force the needle through the sealed end portion and into the body of the user and inject a drug from the drug container assembly through the needle and into the body of the user;
a cap having a solid shell and a handle receiving opening in one end capable of receiving and securing the handle therein with the cover around the handle end opening and a hollow interior cover cavity capable of receiving and shielding the sealed end portion of the auto-injector body therein.

2. The device of claim 1 wherein the cover further comprises a means for attaching the cover to an external object.

3. The device of claim 2 wherein the means for attaching the cover to an external object comprises a key attaching means.

4. The device of claim 2 wherein the means for attaching the cover to an external object comprises a clip.

5. The device of claim 2 wherein the means for attaching the cover to an external object comprises a slot means.

6. The device of claim 1 wherein the drug container further comprises a plunger means capable of forcing a drug from the drug container into the needle and the needle moving means and drug injecting means comprises a spring means having a restraining means to restrain the spring means in a compressed mode between the auto-injector body and the movable needle and attached drug container assembly and the trigger mechanism is capable of releasing the restraining means upon contact of the trigger mechanism with an interior surface of the handle, and the spring means upon release being capable of forcing the needle through the sealed end portion and capable of moving the plunger to force the drug through the needle.

7. The device of claim 6 further comprising a closed end of the handle opposite to the end opening and further comprising a normally extended spring means between the closed handle end and the auto-injector body, the normally extended spring means capable of biasing the auto-injector body so that the sealed end protrudes out of the handle and the normally extended spring means further capable of yielding upon a pressing in of the auto-injector body so that the trigger contacts the closed end of the handle.

8. The device of claim 1 wherein the handle has a recessed ring around its periphery for gripping.
